(19) United States
(12) Patent Application Publication
(10) Pub. No.: US 2025/0213302 A1
(43) Pub. Date: Jul. 3, 2025

(54) METHOD AND APPARATUS FOR DERMATOLOGICAL TREATMENT

(71) Applicant: The General Hospital Corporation, Boston, MA (US)

(72) Inventors: Dieter Manstein, Coral Gables, FL (US); Michael Wang-Evers, Somerville, MA (US)

(73) Assignee: The General Hospital Corporation, Boston, MA (US)

(21) Appl. No.: 18/848,815

(22) PCT Filed: Mar. 23, 2023
(86) PCT No.: PCT/US2023/064849

§ 371 Date: Sep. 19, 2024

Related U.S. Application Data
(60) Provisional application No. 63/323,647, filed on Mar. 25, 2022.

(51) Int. Cl. A61B 18/20; A61B 18/00

(52) U.S. Cl. CPC A61B 18/203; A61B 2018/0047; A61B 2018/00904

(57) ABSTRACT

An apparatus for directing optical energy onto a sample, including: a difference frequency generation (DFG) laser apparatus; a handpiece optically coupled to at least a portion of the DFG laser apparatus by an optical fiber arrangement; and a controller in operative communication with the DFG laser apparatus and the handpiece, wherein the DFG laser apparatus is configured to generate both ablative and nonablative optical energy, and wherein the handpiece includes at least one of an optical or a micromechanical element configured to generate a first pulse and a second pulse of optical energy, wherein a first amount of at least one of ablative optical energy or nonablative optical energy in the first pulse is different from a second amount of at least one of ablative optical energy or nonablative optical energy in the second pulse, and wherein the controller is configured to direct the first pulse and the second pulse onto a particular location on the sample using the handpiece.

### CROSS-REFERENCE TO RELATED APPLICATIONS

The present application is based on and claims priority from U.S. Patent Application Ser. No. 63/323,647, filed on Mar. 25, 2022, the entire disclosure of which is incorporated herein by reference.

### FIELD OF THE DISCLOSURE

The present disclosure relates generally to methods and apparatus for ablating holes in tissue, and more specifically, to providing a plurality of energy pulses directed into the holes having different properties to generate controlled amounts of coagulated tissue within the holes. Such methods and apparatus can be used, e.g., for fractional photothermolysis of skin and other tissues, improved transdermal drug delivery, etc.

### BACKGROUND INFORMATION

Cosmetic procedures and other dermatological treatments that employ a laser to generate small regions of thermal damage or ablation in skin tissue are known in the medical care field for fractional photothermolysis of skin and other tissues. For example, fractional skin resurfacing can relate to a cosmetic procedure where small regions of thermal damage are formed in skin tissue, for example, using electromagnetic energy such as a laser beam.

### SUMMARY

The present disclosure provides systems and methods for ablating a hole in tissue with a plurality of energy pulses directed into the hole having different properties, to generate controlled amounts of coagulated tissue within the hole.

In one embodiment, the disclosure provides an apparatus for directing optical energy onto a sample, including: a difference frequency generation (DFG) laser apparatus; a handpiece optically coupled to at least a portion of the DFG laser apparatus by an optical fiber arrangement; and a controller in operative communication with the DFG laser apparatus and the handpiece, wherein the DFG laser apparatus is configured to generate both ablative and nonablative optical energy, and wherein the handpiece includes at least one of an optical or a micromechanical element configured to generate a first pulse and a second pulse of optical energy, wherein a first amount of at least one of ablative optical energy or nonablative optical energy in the first pulse is different from a second amount of at least one of ablative optical energy or nonablative optical energy in the second pulse, and wherein the controller is configured to direct the first pulse and the second pulse onto a particular location on the sample using the handpiece.

In another embodiment, the disclosure provides a method for directing optical energy onto a sample, including: generating both ablative and nonablative optical energy using an apparatus comprising a difference frequency generation (DFG) laser arrangement and a handpiece optically coupled to at least a portion of the DFG laser arrangement by an optical fiber arrangement, wherein the DFG laser arrangement is configured to generate both ablative and nonablative optical energy, and wherein the handpiece includes at least one of an optical or a micromechanical element configured to deliver a first pulse and a second pulse of optical energy; and generating the first pulse and the second pulse of optical energy using the apparatus; and directing the first pulse and the second pulse onto a particular location on the sample, wherein a first amount of at least one of ablative optical energy or nonablative optical energy in the first pulse is different from a second amount of at least one of ablative optical energy or nonablative optical energy in the second pulse.

### BRIEF DESCRIPTION OF THE DRAWINGS

The disclosure will be better understood and features, aspects, and advantages other than those set forth above will become apparent when consideration is given to the following detailed description thereof. Such detailed description makes reference to the following drawings which may not be drawn to scale.

FIG. 1 is a schematic illustration of a difference frequency generator (DFG) fiber laser according to the present disclosure.

FIG. 2 is a graph of data for ablation in biological tissue for the DFG fiber laser according to the present disclosure in comparison with other lasers.

FIG. 3 shows top-down and cross-sectional images of histological samples showing the effects of the DFG fiber laser on skin tissue according to the present invention in comparison with other lasers.

FIG. 4 is a schematic illustration of a fiber laser apparatus according to the present invention.

FIGS. 5A-5C are illustrations of thermal effects that can be achieved in skin tissue by precise delivery of both ablative and non-ablative optical energy from a laser according to the present invention.

FIGS. 6A-6F are illustrations of apparatus for mechanically stabilizing the tissue in the region of treatment according to the present invention.

FIG. 7 shows an example of a system for directing optical energy onto a biological tissue in accordance with some embodiments of the disclosed subject matter.

FIG. 8 shows an example of hardware that can be used to implement computing device and server in accordance with some embodiments of the disclosed subject matter.

FIG. 9 shows an example of a process for directing optical energy onto a biological tissue in accordance with some embodiments of the disclosed subject matter.

### DETAILED DESCRIPTION

The present disclosure relates to a method and apparatus for directing energy onto biological tissue, e.g., for fractional skin resurfacing, that includes ablating one or more holes in a region of the tissue surface using electromagnetic energy, such as optical energy produced by a laser, then directing further pulses of electromagnetic energy into at least some of the holes to generate further coagulated tissue therein and/or ablate at least a portion of the coagulated tissue that has formed therein. Further pulses can be directed into the same hole to generate additional coagulation and/or remove additional tissue from the holes.

Each region is preferably small, for example, less than 1 mm in diameter or less than 0.5 mm in diameter, and surrounded by substantially unaffected, healthy tissue. The areal fraction of skin surface area covered by damaged tissue after a conventional fractional resurfacing treatment can be typically between about 5% and about 40-50%. Because the regions of tissue damage can be small and separated by healthy tissue, there is a reduced risk of infection or other complications in fractional procedures as compared to procedures such as chemical peels, and healing of the tissue can be faster due to the presence of healthy tissue adjacent to the small regions of damage.

The optical energy pulses can be generally categorized by their typical effects when directed onto biological tissue such as skin. For example, an ablative electromagnetic energy pulse can vaporize tissue and thereby ablate a hole, for example, to remove at least some tissue. A non-ablative electromagnetic energy pulse (an “NA pulse”) can heat tissue locally to coagulate a portion of it, with no associated tissue ablation or vaporization.

The damaged regions can be generated by heating and/or ablation, where ablation can lead to formation of small holes in the tissue as the heated tissue vaporizes and the by-products escape from the hole. The area surrounding the ablated tissue typically includes some coagulated tissue generated by the absorbed energy, where the extent of ablation and local coagulation can depend on parameters of the energy pulse. Such parameters include, e.g., pulse wavelength, pulse duration, pulse intensity, beam diameter, etc.

Non-ablative treatments can produce regions of thermally damaged tissue in the absence of tissue vaporization or removal. Such regions of thermal damage can generate collagen shrinkage, coagulation, and/or a wound healing response that can lead to such effects as an overall tightening of the skin tissue and improved appearance in the treated area. The amount of coagulation produced by a non-ablative pulse can also depend on such parameters of the energy pulses including, e.g., pulse wavelength, pulse duration, pulse intensity, beam diameter, number of pulses, etc.

Such small-scale tissue damage can also be performed on other body tissues besides skin. In any such procedures, it may be desirable to generate both ablative and non-ablative tissue damage. Accordingly, disclosed herein are embodiments of methods, apparatus, and systems for generating controlled amounts of both ablative and non-ablative damage in small regions of tissue, to achieve various beneficial effects including, but not limited to, skin tightening, enhanced absorption of drugs applied transdermally, and the like.

Embodiments of the disclosure provide apparatus, methods, and systems for generating both ablative and non-ablative energy using a fiber laser arrangement, and a handpiece that can be configured to select either ablative or non-ablative energy for each energy pulse directed onto the tissue. The use of a fiber laser arrangement can provide precise spatial accuracy, such that both ablative and non-ablative energy can be directed onto the same tissue location in a short time period.

Unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C.

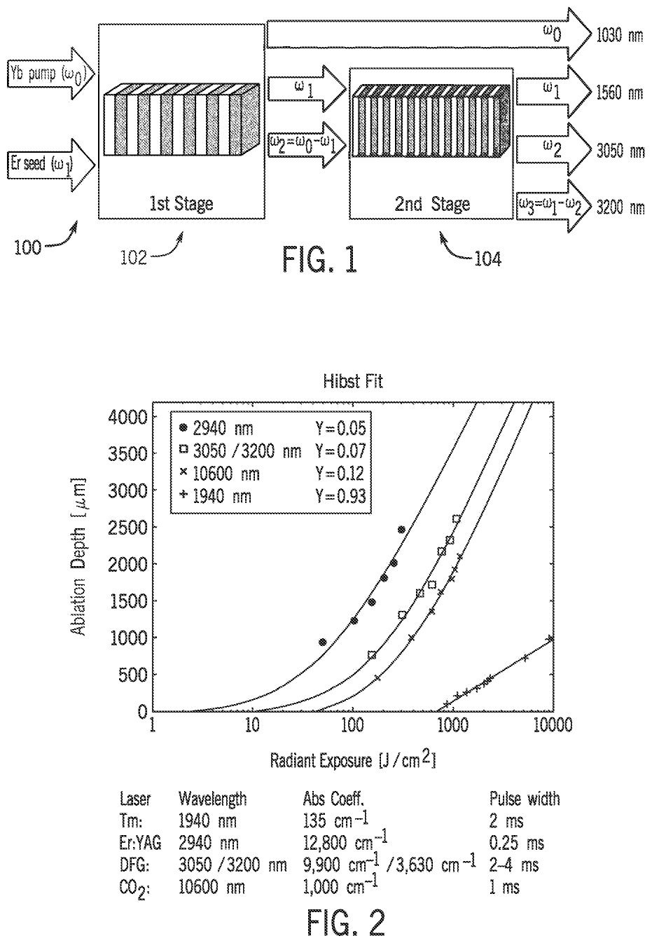

FIG. 1 is a schematic illustration of a difference frequency generator (DFG) fiber laser 100 that can be used with certain embodiments. In some embodiments, the DFG fiber laser 100 is configured to generate four wavelengths of optical energy: 1030 nm, 1560 nm, 3050 nm, and 3200 nm. The former two wavelengths (1030 nm and 1560 nm) tend to produce non-ablative effects in biological tissue, whereas the latter two wavelengths (3050 nm and 3200 nm) can ablate such tissue. A number of variables (e.g., wavelength, energy density, pulse duration, pulse repetition) can be modified to create an ablative or non-ablative laser output energy. In general, the ablation of biological tissue is largely determined by the water absorption coefficient where a high water absorption coefficient will result in ablation while a low water absorption coefficient will result in non-ablative heating. Depending on the energy density and pulse duration of the laser beam there are wavelength ranges that can be used for both ablative and non-ablative treatment. In various embodiments, the non-ablative wavelength range can be defined as ranging from 250 nm to 2,100 nm while the ablative wavelengths are typically within the near to mid infrared spectrum ranging from 1,900 nm to 25,000 nm. However, even longer ablative wavelengths within the far infrared range (25-100 μm) can be achieved with difference-frequency generation.

Difference frequency generation (DFG) in lasers is a nonlinear process that involves combining two photons of different energies to produce a third photon whose energy equals the difference between those of the incident photons. This is based on the fact that the amount of energy in a given photon is directly proportional to the photon's electromagnetic frequency (ω) and inversely proportional to its wavelength (λ) such that the higher the photon's frequency (ƒ), the higher its energy while the longer the photon's wavelength, the lower its energy. As illustrated in FIG. 1, the DFG fiber laser 100 can include and use an Yb (Ytterbium) pump to generate a first frequency ω₀ corresponding to a wavelength of 1030 nm. The DFG fiber laser 100 can also include and use an Er (Erbium) seed to generate a second frequency ω₁ corresponding to a wavelength of 1560 nm. The DFG fiber laser 100 as shown in FIG. 1 has two stages, where in each stage an appropriate crystal (e.g., a first stage crystal 102 and a second stage crystal 104) is used to generate additional frequencies of optical energy where the frequencies represent a difference between the input frequencies. Such crystals, which may be used for DFG and for sum frequency generation (SFG), are known to those skilled in the art and can be selected and adapted for various input frequencies.

For example, the difference between ω₀ and ω₁ in the first stage of the DFG fiber laser 100 shown in FIG. 1, ω₂, corresponds to a wavelength of 3050 nm. In a second stage of the DFG fiber laser 100, a difference between frequencies ω₁ and ω₂, i.e. ω₃, corresponds to an energy output having a wavelength of 3200 nm.

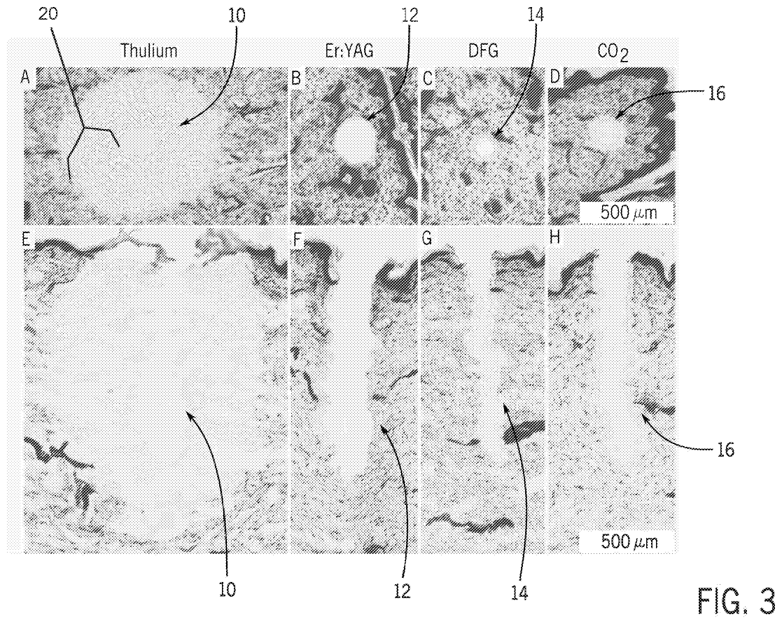

FIG. 2 is a graph showing some exemplary ablation data in biological tissue for the combined 3050/3200 nm wavelengths of the DFG fiber laser 100 as compared to three other ablative lasers: Thulium (Tm) (1940 nm), Er: YAG (2940 nm), and CO2 (10600 nm). As shown in this figure, the Er:YAG has the highest absorption coefficient and produces the greatest ablation depth for a particular intensity, even at shorter pulse durations. The DFG fiber laser 100 is closer in ablative properties to the CO2 laser, having absorption coefficients greater than that for a CO2 laser and producing deeper ablation effects for similar pulse durations at the same intensities (as measured in J/cm²). The Tm laser, by comparison, produces the least amount of ablation, and requires a larger intensity to do so, due in part to its smaller absorption coefficient compared to the other lasers tested.

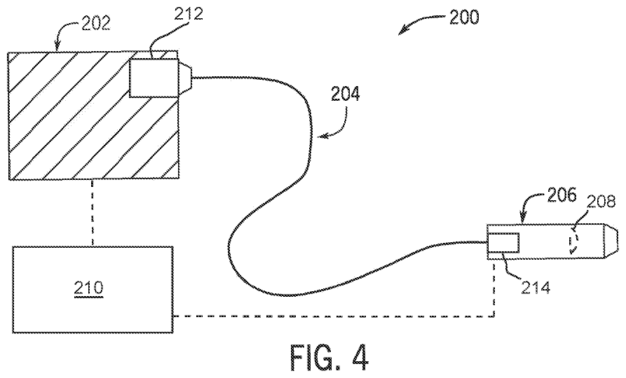

Top-down and cross-sectional images of histological samples showing the effects of several exemplary laser pulses on skin tissue are shown in FIG. 3. FIGS. 3A-3D show surface views of ablated holes 10, 12, 14, 16 using a Thulium laser, an Er:YAG laser, the DFG fiber laser 100, and a CO2 laser, respectively, and FIGS. 3E-H are cross-sectional views of the ablated holes 10, 12, 14, 16 corresponding to FIGS. 3A-3D, respectively. Parameters used to generate the ablated holes 10, 12, 14, 16 were adjusted to generate similar ablation depths, with a spot size of about 100 μm for each laser.

As can be seen in FIGS. 3A and 3E, the Thulium laser produced the greatest amount of coagulated tissue 20 (light area) around the ablated hole. The Er:YAG laser produced the least amount of coagulation around the ablated hole (FIGS. 3B and 3F). The coagulation behavior of the DFG fiber laser 100 (FIGS. 3C and 3G) and the CO2 laser (FIGS. 3D and 3H) are somewhat similar, producing some tissue coagulation around the ablated region. In practice, the Er:YAG laser tends to produce noticeable bleeding when ablating biological tissue, whereas the CO2 laser tends to generate a significant coagulation zone (CZ) around the vaporized tissue that inhibits bleeding. The DFG fiber laser 100, described herein, tends to produce a smaller CZ than a CO2 laser while still being effective at ablating tissue, with the smaller CZ being large enough to avoid unwanted bleeding produced by the Er:YAG laser. Accordingly, the DFG fiber laser 100 with 3050 nm and 3200 nm output wavelengths provides a desirable optical energy output that is effective for ablation while generating a relatively small CZ that is still large enough to inhibit bleeding after ablation.

The DFG fiber laser 100 described herein can also generate non-ablative optical energy beams having wavelengths of 1030 nm and 1560 nm. Such energy, when focused onto biological tissue, can thermally damage the tissue to produce coagulation without ablating it. Controlled amounts of tissue coagulation can be desirable for a variety of effects. For example, coagulation of skin tissue can promote skin tightening and collagen shrinkage, which may improve the cosmetic appearance. Coagulation in subsurface tissues surrounding ablated holes can also promote penetration and absorption of hydrophilic and/or low molecular weight (MW) drugs and compounds. Such applications and benefits are described in more detail below.

In some dermatological and other biological laser procedures, it may be desirable to reduce the amount of coagulation surrounding ablated holes, and/or to avoid excessive bleeding. In other applications, it may be desirable to control or increase the size of the CZ around an ablated hole. Accordingly, embodiments of the disclosure can provide a method and apparatus for ablating small holes in tissue and tailoring the amount of coagulation around the ablated region by using both ablative and non-ablative wavelengths of the optical energy generated by a laser such as the DFG fiber laser 100 described herein that can generate both ablative and non-ablative optical energy in rapid succession and deliver the energy to the tissue via a single fiber arrangement for spatial consistency. In some embodiments, the optical output of the DFG fiber laser 100 can be switched controllably and rapidly between ablative and non-ablative frequencies. Additionally, or alternatively, such frequencies can be emitted simultaneously in controlled ratios. In various embodiments, the diameter of the light beam and tissue removal or photothermal destruction (coagulation zone) can be in a range of 10-1000 μm, and preferably in the range of 30 to 300 μm. The desired ablation depth can be in the range of 10 μm to 5 mm.

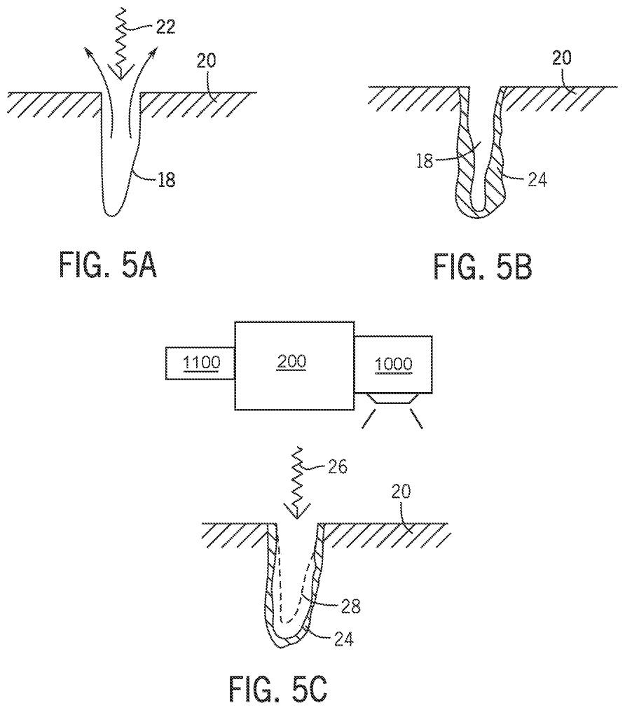

Accordingly, in certain embodiments of the disclosure, a fiber laser apparatus 200 can be provided such as, for example, that illustrated schematically in FIG. 4. The exemplary apparatus of FIG. 4 includes a laser unit 202 capable of generating both ablative and non-ablative wavelengths of optical energy, such as, for example, the DFG fiber laser 100 described above, a fiber 204, and a handpiece 206. The fiber 204 is configured to convey optical energy produced from the laser unit 202 to the handpiece 206. The handpiece 206 can optionally include certain components of the laser unit 202 such as, for example, the first and second stage crystals 102, 104 of the DFG fiber laser 100 shown in FIG. 1. The first and second stage crystals 102, 104 receive the optical energy output from the Yb pump and Er seed of the laser unit 202 and produce desired wavelengths of optical energy based on frequency difference generation. In various embodiments the apparatus 200 may include a controller 210 which may be in communication (e.g. wired and/or wirelessly) with one or both of the laser unit 202 and/or the handpiece 206 (see FIG. 4). Furthermore, some or all of the controller 210 may be integrated into one or both of the laser unit 202 and/or the handpiece 206. In certain embodiments, the controller 210 may be configured to carry out one or more of the functions of the apparatus 200 that are disclosed herein.

The handpiece 206 can further include conventional optical elements 208 such as lenses that can be configured to direct and/or focus the optical energy generated by the laser unit 202 (e.g., the DFG fiber laser 100 or portions thereof) from the handpiece 206 onto a target (e.g., skin or other tissue), where such focusing can include controlling the spot size and/or convergence/divergence of the emitted optical beam.

The fiber laser apparatus 200 of FIG. 4 can also include switching elements 212, 214 (such as optical or micromechanical elements, e.g., as part of the laser unit 202 and/or the handpiece 206, respectively; see below and FIG. 4) with further optical components, and optionally mechanical components, which are configured to selectively change the output of the fiber laser apparatus 200 between ablative and non-ablative wavelengths, and/or to provide an optical energy output that includes certain combinations of such wavelengths. For example, such switching elements can be configured to selectively reflect, transmit, block, attenuate, and/or filter certain optical wavelengths to vary the output between ablative and non-ablative optical beams.

In some embodiments, the two types of optical energy can be split between the first stage and the second stage of a laser apparatus such as the DFG fiber laser 100 shown in FIG. 1. For example, the beam path can be altered to divert the path of at least a portion of the 1030 nm and 1560 nm beams before each hits the second stage crystal 104 and direct this portion directly to the fiber output (e.g., the handpiece 206 of the fiber laser arrangement 200), thus providing an output of non-ablative energy. Such diversion of the beam path can selectively be turned off, thereby allowing output of the 3050 nm and 3200 nm ablative wavelengths from the second stage crystal 104. Similarly, the non-ablative 1560 nm wavelength can be selectively filtered or reflected from the output of the first stage crystal 102 such that only the ablative wavelengths are conveyed through the fiber 204 to the handpiece 206 and onto a target.

Rapid switching between ablative and non-ablative outputs can be achieved through the use of techniques and mechanisms known in the art (e.g., using optical or micromechanical switching elements 212, 214, which may be part of the laser unit 202 and/or the handpiece 206, respectively, or may be separate components) that include appropriate wavelength-dependent optical elements and shifting them electronically. In one embodiment, a polarizer and a polarizing beam splitter can be used to separate the two types of wavelengths. Polarization of incoming light (optical energy) can be electronically switched by using optical elements such as, for example, liquid crystals, electro-optical modulators (e.g. Pockels cells), or Faraday rotators. Polarization-based shutters rotate a polarization element between two linear filters and tend to have moderate switching speeds, on the order of tens of milliseconds, but can operate on low power and do not have precise alignment requirements for operation.

In further embodiments, rapid output selection between ablative and non-ablative optical energy can be achieved using, for example, reflection- or diffraction-based switching arrangements or elements. Such arrangements can generate pulses of optical energy by employing electronically-driven (e.g. micromechanical) optical elements that reflect or diffract the optical energy based on its frequency/wavelength, such that portions of the laser output can be directed in different optical paths based on their wavelengths. The electronically-driven optical elements can include, for example, acousto-optic and electro-optic modulators, flip/movable mirrors, electrical actuators, galvanometer scanners, rotation stages, and/or spinning mirror drums. Acousto-optic and electro-optic modulators can be very fast, providing switching on the order of a microsecond or less, and are highly tunable for different wavelengths. Acousto-optic modulators use a pressure wave diffraction grating to shift laser frequencies and produce several orders of spatially separated diffracted beams, where particular beams can then be selected using spatially oriented shuttering arrangements.

In further embodiments, an electronic shutter or optical choppers may be employed to open/close light paths of the two types of energy after they have been separated. Mechanical shutters can operate very quickly and can provide 100% switching between transmission and blocking of optical beams.

In these embodiments, the optical and/or micromechanical elements used to separate ablative and non-ablative energy and direct them to a fiber output can be specified based on the particular wavelengths of interest. Although the exemplary DFG fiber laser 100 shown in FIG. 1 is configured to emit four particular wavelengths, embodiments of the disclosure can employ any such laser that is configured to produce at least one wavelength of ablative energy and at least one wavelength of non-ablative energy. For example, in a further embodiment, a laser arrangement may be used that includes a pump wavelength (ω₀) of 1030 nm and a seed wavelength (ω₁) of 1440 nm (both of which are non-ablative), resulting in combined ablative output wavelengths ω₂, ω₃ of 3600 nm and 2400 nm, respectively. In yet another embodiment, the laser arrangement may be used that includes a pump wavelength (ω₀) and a seed wavelength (ω₁) of 1064 nm and 1560 nm respectively (both non-ablative), resulting in the combined ablative output wavelengths ω₂ of 3350 nm and ω₃ of 2900 nm. Other laser arrangements having nonablative pump and seed wavelengths may also be used, where such wavelengths can be combined using DFG principles to produce further output wavelengths that are ablative.

Exemplary ablative/non-ablative laser systems as described herein can be provided with conventional control systems (e.g. controller 210) to select, vary, and control certain aspects of their operation. For example, controls can be provided on the handpiece or laser unit (for example the handpiece 206 or the laser unit 202) to switch between ablative and non-ablative outputs and can be designed based on the particular type of wavelength-switching arrangement used. In some embodiments, the system can be configured to generate predetermined sequences of optical energy pulses, where parameters that may be controlled included pulse duration, pulse intensity or energy, pulse timing, and wavelength(s) of the emitted pulses (e.g., ablative or non-ablative). Because of the ability to rapidly switch between types of emitted optical energy and having such energy delivered through an optical fiber waveguide (e.g., the fiber 204) to the handpiece 206, such laser systems can provide high spatial consistency such that the optical energy (e.g. in the form of a plurality of pulses) can be directed onto one or more target locations with high precision. The importance of such spatial precision can be appreciated based on the below description of exemplary operation of such devices.

Controls can also be provided (e.g. by controller 210) for varying the geometric properties of the emitted energy. For example, a plurality of conventional lens arrangements can be provided in the handpiece 206, such that optical beams having different beam widths, focal lengths, and divergent/convergent angles can be provided. Such optical controls are known in the art for fiber lasers and can include preprogrammed and/or manual selection of beam geometry parameters.

In some implementations, devices or systems disclosed herein can be utilized, manufactured, installed, etc. using methods embodying aspects of the invention. Correspondingly, any description herein of particular features, capabilities, or intended purposes of a device or system is generally intended to inherently include disclosure of a method of using such devices or systems for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated or limited, discussion herein of any method of manufacturing or using a particular device or system, including installing the device or system, is intended to inherently include disclosure, as embodiments of the invention, of the utilized features and implemented capabilities of such device or system. For example, some embodiments of the present disclosure can provide methods and apparatus for forming a small ablated hole (e.g., less than 1 mm in diameter, or less than about 0.5 mm) in a target tissue using one or more pulses of ablative energy, and then directing one or more further optical pulses into the holes to generate further tissue coagulation, ablate a portion of the coagulated tissue, and/or to ablate further tissue deeper than the existing hole. The parameters of such pulses (e.g., pulse type (ablative/non-ablative), pulse energy or intensity, pulse duration, timing between pulses, beam width and geometry, etc.) can be determined based on the desired effects to be produced in the tissue.

In general, ablative energy pulses having higher power and shorter duration tend to more strongly ablate tissue, vaporizing a portion thereof based on the large amount of energy that is absorbed by the tissue in a short time (e.g., shorter than a thermal relaxation period). For such ablative pulses, the energy tends to heat up tissue quickly enough to vaporize a portion thereof. These highly ablative pulses tend to have a very thin thermal affected zone (which may, e.g., include a coagulated zone) around the ablated volume. In contrast, pulses having lower power and longer durations tend to heat up tissue more slowly, such that some energy can be dissipated to surrounding tissue and generate more tissue coagulation, protein denaturation, etc. A single pulse can both ablate and coagulate tissue. Also, a laser pulse having a particular set of parameters may affect different tissues differently, e.g., based on the chromophores present, tissue structures may be present in the tissue, etc. For example, the energy generated by an ablative laser such as a CO2 laser tends to be strongly absorbed by water and thus its effect on tissue may depend more on the amount of water present in the tissue than on the presence or absence of visible chromophores such as pigments.

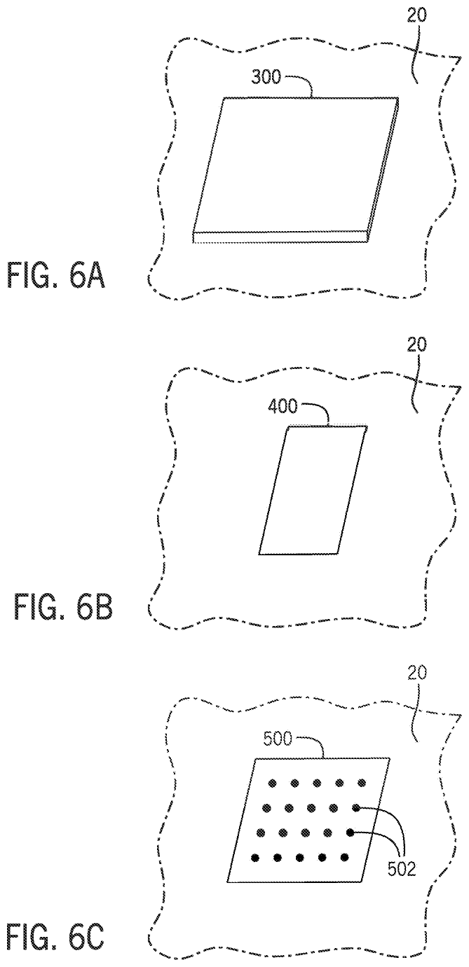

A simplified illustration of thermal effects that can be achieved in skin tissue by precise delivery of both ablative and non-ablative optical energy (e.g. using the) is shown in FIGS. 5A-5C. FIG. 5A shows a typical cross-sectional view of a hole 18 being formed in skin 20 or other tissue by application of one or more pulses of directed ablative energy 22. The width and depth of the ablated hole 18 can depend on a variety of parameters including, e.g., tissue type, parameters of the energy beam, etc. Histograms of some actual ablated holes (which are shown in FIGS. 3A-3H) formed by different types of lasers are illustrated in FIG. 2.

There is typically a zone of heat-affected, coagulated tissue around the surface of an ablated hole. FIG. 5B shows a (sometimes thin) zone of coagulated tissue 24 that can be formed around the ablated hole 18. Coagulation can tend to contract and/or shrink the tissue, which may contain collagen or other structural proteins. Further, tissue proximal to the ablated hole 18 may also be heated or otherwise react to the applied energy 22, such that it may expand and partially refill the lower portion of the ablated hole 18. The time for coagulation to occur after exposure of tissue to an energy beam (e.g. a laser) can be very short, for example, on the order of milliseconds or tens of milliseconds.

In some embodiments, a second ablative laser pulse 26 can be directed into the ablated hole 18, as shown in FIG. 5C, to ablate at least a portion of the coagulated tissue 24 that has formed and contracted within the ablated hole 18 shown in FIG. 5B. For example, a removed/ablated portion 28 is shown in FIG. 5C between a former external boundary of the coagulated tissue 24 (i.e., the dashed line) and a new external boundary of the coagulated tissue 24 (i.e., the solid line spaced inward of the boundary of the ablated hole 18). This procedure allows for more tissue removal and a greater degree of shrinkage within a single ablated micro-hole by directing a plurality of pulses into the ablated hole 18. The second pulse 26, and/or further pulses can be provided after a suitable time interval that allows local coagulation around the ablated hole 18 to occur.

Additionally, one or more non-ablative pulses can be directed into the ablated hole 18 such that, after the ablated hole 18 is formed, additional coagulated tissue can be generated within the ablated hole 18 (not illustrated). In another embodiment, the coagulation and ablation steps can be alternated, such that deeper holes surrounded by additional coagulated tissue can be formed. Any desired combination of pulses can be directed onto a single location, to controllably and precisely form ablated holes and coagulation zones within and adjacent to such holes. Parameters for such pulse sequences that include both ablative and non-ablative pulses can be selected based on known properties of the tissue interactions with various types of optical energy. In various embodiments, the delivery of optical energy can be conducted by an embodiment of the fiber laser apparatus 200 operating in conjunction with an optical imaging arrangement 1000 and a position controller 1100 (FIG. 5C), as discussed further below.

In some embodiments, the width and/or convergence/divergence angle of one or more pulses in a series of pulses can be varied to further tailor the local effects of directing optical energy onto the tissue. For example, a narrower or converging non-ablative pulse can follow an ablative pulse to generate more coagulation near the bottom of the ablated hole. Alternatively, a wider non-ablative pulse can be applied to generate more coagulation around the hole, e.g., closer to the surface region. Such increased coagulation near the tissue surface may produce desirable effects such as, e.g., increased tightening and/or faster healing when used in fractional resurfacing procedures.

In further embodiments, the fiber laser apparatus 200 as described herein can be used to first irradiate a target location with one or more pulses of non-ablative optical energy to generate a zone of tissue coagulation in the irradiated volume. This may be followed by one or more pulses of ablative energy directed onto the same location after a short time interval to form an ablated hole within the coagulated tissue. The interval between non-ablative and ablative pulses can be selected, for example, based on the energy, width, and/or intensity of the different pulses, and the desired degree of local coagulation between pulses. General tissue/energy interactions are well-characterized for many types of optical energy, such that the different pulse parameters and timing of pulse sequences can be estimated to achieve desired local ablation and coagulation results without undue experimentation. In some embodiments, a sequence may be used, for example, to first form a thermally-damaged volume of coagulated tissue having a desired size and depth, and then forming a hole in this region to facilitate access to the deeper portions of the coagulated volume. The ablated hole may be used, for example, to improve absorption of certain compounds in a laser-assisted drug delivery procedure, or provide a less-obstructed pathway for further pulses of ablative and/or non-ablative laser pulses.

In some embodiments, pulse sequences that include both ablative and non-ablative pulses can be utilized such that, for example, successive pulses of ablative energy deepen the hole, with non-ablative pulses used to generate additional coagulation within the hole. In this manner, shrinkage, coagulation and/or other tissue effects can be generated at progressively deeper levels within the tissue. Such increase in the amount of tissue coagulation around a single hole may, for example, improve the skin tightening efficacy when used in fractional resurfacing procedures.

In some embodiments, the fiber laser apparatus 200 can be configured to provide pulses that combine ablative and non-ablative optical energy, for example, a single pulse containing energy having a wavelength of ω₀ and/or ω₁, and also containing energy having a wavelength of ω₂ and/or ω₃. The generation of such “mixed” pulses can be achieved, e.g., by using any of the various known techniques and further optical and/or micromechanical arrangements provided, for example, in the handpiece 206 to selectively reflect, transmit, block, attenuate, and/or filter certain optical wavelengths in the output path of the DFG fiber laser 100. The energy of each wavelength in the mixed pulse can be selected, for example, based on such techniques and arrangements adapted to modify the output level for one or more wavelength components of the overall output of the DFG fiber laser 100.

Mixed pulse sequences that include certain pulses each having both ablative and non-ablative optical energy, and optionally further pulses having either ablative or non-ablative energies, can be utilized to provide even more varied and tailored zones of coagulated and ablated tissue in a target region. Relative amounts of ablation, shrinkage, coagulation, and/or other tissue effects can be generated within a tissue region by appropriate selection of pulse sequence parameters, where the particular sequence will depend on the desired local effects in the tissue.

The interval between successive pulses can be determined based on several factors. In general, the interval between pulses should be long enough to allow most of the resulting coagulation of tissue to occur. Such coagulation can form in several or tens of milliseconds, for example, based on considerations of local thermal relaxation times. Accordingly, intervals between successive pulses applied to the same location can be, for example, tens of milliseconds, e.g., 20-30 ms or more, up to 100 ms or greater. Relatively smaller intervals may lead to more local preheating as the tissue has less time to cool off between pulses. This cumulative preheating can be controlled to generate increased coagulation and/or ablation with subsequent pulses that have the same or reduced intensity, power, etc. Setting appropriate pulse intervals can also vary the relative amounts of coagulation and ablation that are generated, e.g., by a plurality of alternating ablative and non-ablative pulses, each pulse type having constant pulse properties.

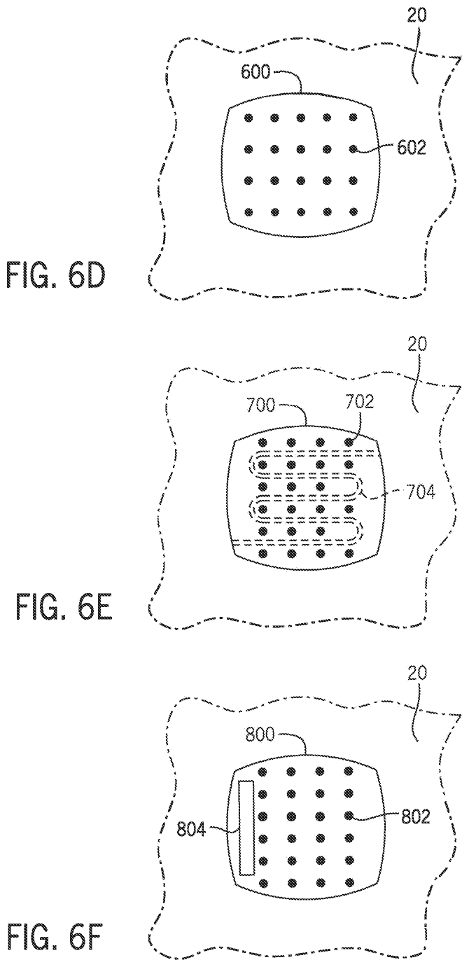

In some embodiments, it may be desirable to mechanically stabilize the tissue surface where one or more holes are ablated and coagulated tissue is being formed by applying a stabilizer such as a plate, a film, or a mask onto the sample prior to directing energy at the sample. FIG. 6A shows that such stabilization can be achieved, for example, by providing a rigid or semi-rigid plate 300 on the tissue surface 20 to inhibit surface movement or deformation of the tissue 20 in the target region. The plate is preferably transparent or weakly absorbent of optical energy having wavelengths emitted by the fiber laser apparatus 200. Alternatively, the plate 300 may be ablatable by the optical energy used to ablate holes in the tissue 20, such that application of ablative energy can form holes that pass through the plate 300 and into the tissue 20. Such tissue stabilization can facilitate precise location of the applied optical energy on the tissue 20, for example, for the duration of a series or sequence of pulses, and it may also inhibit closure of an ablated hole to facilitate directing of further pulses into the hole.

In some embodiments, stabilization may be achieved by applying an adhesive tape 400 or film or the like to the tissue surface 20 prior to application of optical energy as shown in FIG. 6B. Such tape 400 can be transparent to the applied energy, or it may be ablatable such that ablative pulses can form a hole through the tape/film 400 and into the underlying tissue 20. In some embodiments, the tape/film 400 can be applied while the target region of tissue 20 is held under mild or moderate tension, which can further inhibit closure of ablated holes while directing a plurality of optical pulses onto a particular location. Additional features, benefits, and considerations relating to tissue stabilization for such procedures are described, for example, in U.S. Pat. No. 10,092,354, which is hereby incorporated by reference in its entirety along with all related applications and patents.

Embodiments of the disclosure can be used to produce ablated holes in tissue having controllable or selectable widths, depths, and thickness profiles of coagulated tissue within, along the depth of, and/or surrounding the ablated holes. Such control over characteristics of the ablated holes can be used to achieve a variety of results and effects in biological tissues. One example is ablative fractional skin resurfacing, where a plurality of small holes (e.g., holes having a width of about 0.5 mm or less) are ablated in a region of skin tissue. This well-known procedure can achieve a degree of skin tightening and wrinkle reduction through a healing response and physical collagen shrinkage resulting from the thermal damage to the skin. Because fractional resurfacing generates small regions of damage surrounded by healthy tissue, the damage effects tend to be well-tolerated and produce a low risk of infection compared to other procedures such as chemical peels.

The fiber laser apparatus 200 as described herein does not require any mirrored laser arm, and the optical energy is delivered through an optical fiber arrangement (e.g., the fiber 204) to produce the emitted wavelengths via a single handpiece (e.g., the handpiece 206). This configuration facilitates precise and consistent delivery of a focused laser beam into predefined locations with high locational precision in a stable and reproducible manner. Accordingly, some embodiments of the disclosure can provide a precise delivery of the laser focused beams having different wavelengths and properties (e.g. ablative and non-ablative) through a prefabricated mask 500 containing a plurality of openings or holes 502 as shown in FIG. 6C, where the delivered wavelengths can be quickly changed or switched such that multiple wavelengths can simultaneously and alternatingly be delivered to a single precise target location. The mask 500 can be placed over, and optionally adhered to, a target region of tissue 20 and the fiber laser arrangement can direct different types of optical energy through a single hole of the plurality of holes the mask 500 in rapid succession with a high degree of spatial precision. Such spatial precision and rapid switching is not feasible using conventional dual lasers that require moving arms and the like.

In some embodiments as shown in FIGS. 6D through 6F, a mask 600, 700, 800 placed over a target area of tissue 20 can be provided that includes a plurality of openings or holes 602, 702, 802 and a passive cooling arrangement through pre-application preparation as with the mask 600 or with an active cooling arrangement 704, 804 as with the masks 700, 800. As described above, the mask 600, 700, 800 can be placed over or adhered to a target region of tissue. In some embodiments, for example, the mask 600 shown in FIG. 6D can be formed of a material having a high thermal capacity and can be precooled, such that the mask. 600 cools the target region of tissue 20 while in contact therewith, for example, during the application of optical laser energy to the tissue 20 beneath the mask 600. Such passive cooling can provide local analgesia to reduce any pain or discomfort felt by a subject during the laser treatment.

In some embodiments, for example, the mask 700 shown in FIG. 6E can comprise a plurality of holes 702 that can be actively cooled. The mask 700 can be cooled, for example, by circulating a liquid coolant through channels 704 provided within and/or on a surface of the mask 700.

In some embodiments, for example, the mask 800 shown in FIG. 6F can be formed at least partially of a thermally conductive material (such as a metal), and a thermoelectric cooler 804 or other cooling arrangement can be provided in thermal contact with the mask 800. Cooling arrangements for devices such as dermatological masks are known in the art and these and others not disclosed can be used alone or in combination in conjunction with embodiments of the fiber laser apparatus 200 or the DFG fiber laser 100 as disclosed herein. Such active cooling of the mask may generally provide longer durations and more precise control of tissue cooling as compared to passive cooling, and may be used for laser procedures having a longer duration or for which precise cooling conditions are desired.

In some embodiments of the disclosure, various combinations of beam parameters can be used to achieve improved fractional resurfacing cosmetic effects. For example, ablative energy can be applied to one or more target locations to generate ablated holes. Subsequently, one or more non-ablative beam pulses can be directed precisely onto/into the ablated holes to generate additional coagulated tissue within the ablated holes. Optionally, further ablative energy can be applied to the same location, for example, to deepen the ablated hole, and further pulses of non-ablative energy can be directed into the ablated hole to produce even more coagulation deeper in the tissue. This can lead to greater overall shrinkage or tightening effects and/or wrinkle removal based on the same number of ablated surface spots that may be generated in a conventional ablative fractional resurfacing procedure. Additionally, an ablated hole may be filled to a substantial degree by increasing the tissue volume within the initial hole (using non-ablative pulses) more than the volume is reduced from tissue removal by further ablative pulses. Any desired sequence of ablative and non-ablative pulses can be applied onto a precise target location in rapid succession to achieve the desired thermal effects in the tissue.

After one or a plurality of holes are treated with a plurality of pulses as described herein, further holes can be formed in regions proximal to the treated area, and a plurality of pulses directed into these additional holes. The procedure can be repeated until the entire target area has been treated. Geometrical parameters for initial ablative hole sizes and hole patterns/spacings that are used in conventional ablative fractional resurfacing procedures may also be used in embodiments of the present invention.

In some embodiments of the disclosure, the system and apparatus described herein (e.g., the fiber laser apparatus 200) can be used for a variety of cosmetic and therapeutic procedures. For example, perforating tissue with ablated holes can modify or improve the absorption of certain compounds. Such effects form the basis for laser-assisted drug delivery methods and systems. Embodiments of the present disclosure can improve such methods by facilitating greater control of hole geometry and the extent of tissue coagulation within and around the ablated holes.

For example, hydrophilic and low molecular weight (MW) compounds can more easily diffuse through coagulated tissue zones (CZs; for example the zones of coagulated tissue 20 shown in FIGS. 5B and 5C). Examples of such compounds include, e.g., Imiquimod (a topical anti-tumor medication) and Minoxidil and Finasteride (which are used to treat male pattern baldness and hair loss). Accordingly, embodiments of the disclosure can be used to generate ablated holes with certain geometries and tailored CZs to improve the absorption characteristics and efficacy of such compounds.

In some embodiments, the fiber laser apparatus 200 described herein can be combined with an optical imaging arrangement 1000 (see FIG. 5C) and position controller 1100 (which may be coupled to or integrated into the fiber laser apparatus 200, for example with the handpiece 206) to produce ablated holes in tissue at certain locations determined by the imaging arrangement 1000. For example, the imaging arrangement 1000 can be used in conjunction with the fiber laser apparatus 200 and the position controller 1100 to generate ablated holes in the scalp or other body areas that avoid hair follicles, for subsequent application of compounds like Minoxidil and Finasteride. While it is desirable to improve the delivery and absorption of such compounds using small ablated holes, it is preferable to avoid damaging healthy hair structures when forming these holes. Accordingly, the combination of an imaging/positioning arrangement with the precise spatial delivery of optical energy using the fiber laser apparatus 200 can achieve both benefits simultaneously, e.g., improving absorption of these compounds while avoiding damage to existing hair follicles.

Laser-ablated holes can also facilitate the penetration and absorption of high MW drugs and compounds, such as hyaluronic acid and human growth hormone (HGH). However, thick CZs can inhibit the absorption of such compounds by the surrounding tissue. Accordingly, embodiments of the disclosure can provide for formation of ablated holes in tissue having small CZs, or holes having desired CZ profiles, to improve absorption of high MW compounds as compared to holes ablated using, e.g., a CO2 laser or the like, where the CZs tend to be thicker. For example, embodiments of the present disclosure may be used in laser-assisted drug delivery procedures to generate ablated holes with tailored CZ profiles to inhibit fast absorption of certain therapeutic compounds therethrough, where such absorption may then occur over a longer period of time and/or with lower instantaneous levels in the surrounding tissue. Using embodiments of the present disclosure, the characteristics of ablated and coagulated tissues (e.g., hole width and depth, coagulation zone thickness profile along the hole, etc.) may be tailored to provide desired local delivery and absorption rates for certain therapeutic compounds and the like.

The systems and methods described herein facilitate the formation of precisely-located ablated holes in tissue with surrounding coagulation zones that can be tailored by selection of both ablative and non-ablative wavelengths, which can be provided as pulses of optical energy. The parameters of these pulses can also be selected to further tailor the characteristics of the ablated holes for particular treatments and procedures. Such pulses can be generated and emitted in rapid sequences with short intervals between them, and delivering such energies through a fiber arrangement allows precise spatial precision during the application of a plurality of pulses. Accordingly, in addition to the few practical examples described herein, embodiments of the present disclosure may provide useful for other types of laser-based procedures performed on various body parts, such as endoscopic applications, including but not limited to: neurological surgery, oral surgery, cardiovascular surgery, oncology, gastrointestinal surgery, cataract surgery, and other dermatological or non-dermatological procedures.

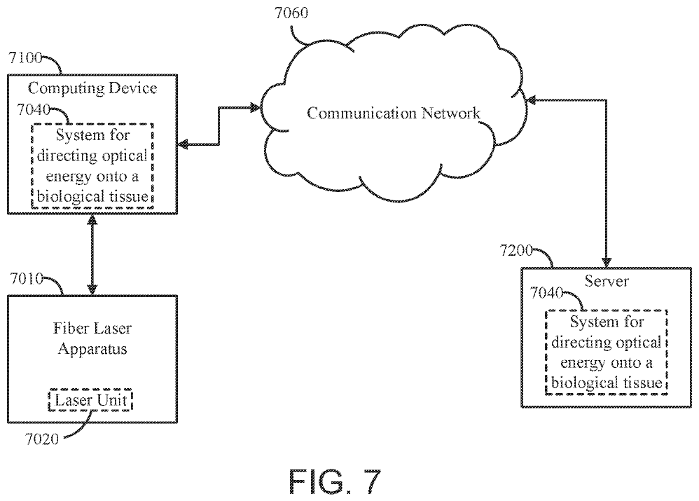

Turning to FIG. 7, an example 7000 of a system (e.g. a tissue treatment system) for directing optical energy onto a biological tissue is shown in accordance with some embodiments of the disclosed subject matter. As shown in FIG. 7, a computing device 7100 can send and receive control information to a fiber laser apparatus 7010. In some embodiments, computing device 7100 can execute at least a portion of a system for directing optical energy onto a biological tissue 7040 to perform dermatological treatment of the biological tissue. Additionally or alternatively, in some embodiments, computing device 7100 can communicate information about fiber laser apparatus 7010 to a server 7200 over a communication network 7060, which can execute at least a portion of system for directing optical energy onto a biological tissue 7040 to perform dermatological treatment of the biological tissue. In some such embodiments, server 7200 can return information to computing device 7100 (and/or any other suitable computing device) indicative of an output of system for directing optical energy onto a biological tissue 7040, such as information for generating ablative or non-ablative pulses of optical energy. Information from the system may be transmitted and/or presented to a user (e.g. a researcher, an operator, a clinician, etc.) and/or may be stored (e.g. as part of a research database or a medical record associated with a subject).

In some embodiments, computing device 7100 and/or server 7200 can be any suitable computing device or combination of devices, such as a desktop computer, a laptop computer, a smartphone, a tablet computer, a wearable computer, a server computer, a virtual machine being executed by a physical computing device, etc. As described herein, system for directing optical energy onto a biological tissue 7040 can present information related to the system, e.g. information for generating ablative or non-ablative pulses of optical energy, to a user (e.g., researcher and/or physician).

In some embodiments, fiber laser apparatus 7010 may include a laser unit 7020, which can be any laser unit suitable for generating ablative or non-ablative optical energy pulses. In other embodiments, laser unit 7020 can be local to computing device 7100. For example, laser unit 7020 may be incorporated with computing device 7100 (e.g., computing device 7100 can be configured as part of a device for directing optical energy into a biological tissue). As another example, laser unit 7020 may be connected to computing device 7100 by a cable, a direct wireless link, etc. Additionally or alternatively, in some embodiments, laser unit 7020 can be located locally and/or remotely from computing device 7100, and can communicate (send or receive) information to computing device 7100 (and/or server 7200) via a communication network (e.g., communication network 7060).

In some embodiments, communication network 7060 can be any suitable communication network or combination of communication networks. For example, communication network 7060 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 7060 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, etc.

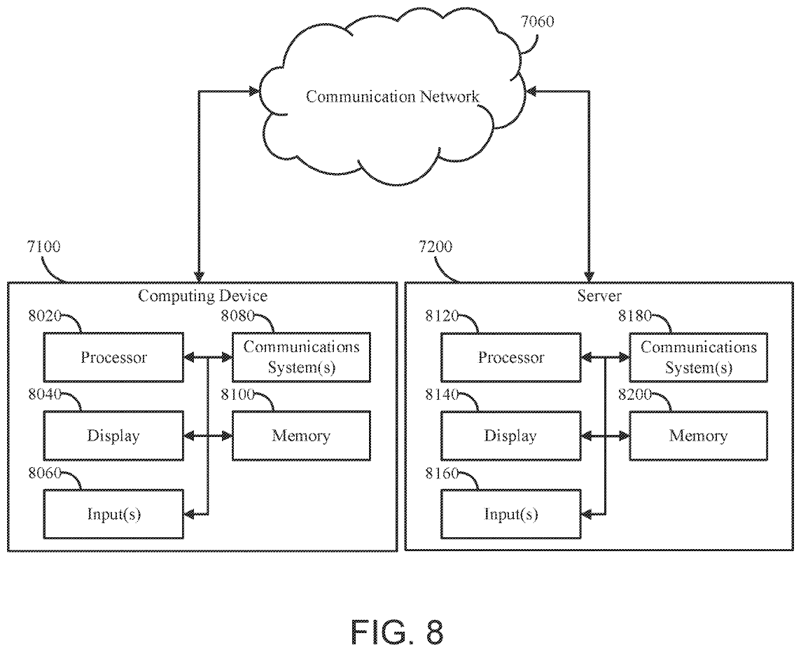

FIG. 8 shows an example 8000 of hardware that can be used to implement computing device 7100 and server 7200 in accordance with some embodiments of the disclosed subject matter. As shown in FIG. 8, in some embodiments, computing device 7100 can include a processor 8020, a display 8040, one or more inputs 8060, one or more communication systems 8080, and/or memory 8100. In some embodiments, processor 8020 can be any suitable hardware processor or combination of processors, such as a central processing unit, a graphics processing unit, etc. In some embodiments, display 8040 can include any suitable display devices, such as a computer monitor, a touchscreen, a television, etc. In some embodiments, inputs 8060 can include any suitable input devices and/or sensors that can be used to receive user input, such as a keyboard, a mouse, a touchscreen, a microphone, etc.

In some embodiments, communications systems 8080 can include any suitable hardware, firmware, and/or software for communicating information over communication network 7060 and/or any other suitable communication networks. For example, communications systems 8080 can include one or more transceivers, one or more communication chips and/or chip sets, etc. In a more particular example, communications systems 8080 can include hardware, firmware and/or software that can be used to establish a Wi-Fi connection, a Bluetooth connection, a cellular connection, an Ethernet connection, etc.

In some embodiments, memory 8100 can include any suitable storage device or devices that can be used to store instructions, values, etc., that can be used, for example, by processor 8020 to present content using display 8040, to communicate with server 7200 via communications system(s) 8080, etc. Memory 8100 can include any suitable volatile memory, non-volatile memory, storage, or any suitable combination thereof. For example, memory 8100 can include RAM, ROM, EEPROM, one or more flash drives, one or more hard disks, one or more solid state drives, one or more optical drives, etc. In some embodiments, memory 8100 can have encoded thereon a computer program for controlling operation of computing device 7100. In such embodiments, processor 8020 can execute at least a portion of the computer program to present content (e.g., images, user interfaces, graphics, tables, etc.), receive content from server 7200, transmit information to server 7200, etc.

In some embodiments, server 7200 can include a processor 8120, a display 8140, one or more inputs 8160, one or more communications systems 8180, and/or memory 8200. In some embodiments, processor 8120 can be any suitable hardware processor or combination of processors, such as a central processing unit, a graphics processing unit, etc. In some embodiments, display 8140 can include any suitable display devices, such as a computer monitor, a touchscreen, a television, etc. In some embodiments, inputs 8160 can include any suitable input devices and/or sensors that can be used to receive user input, such as a keyboard, a mouse, a touchscreen, a microphone, etc.

In some embodiments, communications systems 8180 can include any suitable hardware, firmware, and/or software for communicating information over communication network 7060 and/or any other suitable communication networks. For example, communications systems 8180 can include one or more transceivers, one or more communication chips and/or chip sets, etc. In a more particular example, communications systems 8180 can include hardware, firmware and/or software that can be used to establish a Wi-Fi connection, a Bluetooth connection, a cellular connection, an Ethernet connection, etc.

In some embodiments, memory 8200 can include any suitable storage device or devices that can be used to store instructions, values, etc., that can be used, for example, by processor 8120 to present content using display 8140, to communicate with one or more computing devices 7100, etc. Memory 8200 can include any suitable volatile memory, non-volatile memory, storage, or any suitable combination thereof. For example, memory 8200 can include RAM, ROM, EEPROM, one or more flash drives, one or more hard disks, one or more solid state drives, one or more optical drives, etc. In some embodiments, memory 8200 can have encoded thereon a server program for controlling operation of server 7200. In such embodiments, processor 8120 can execute at least a portion of the server program to transmit information and/or content (e.g., results of a tissue identification and/or classification, a user interface, etc.) to one or more computing devices 7100, receive information and/or content from one or more computing devices 7100, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

In some embodiments, the optical signals are detected by photodiodes. It should be recognized that any opto-electronic conversion device including but not limited to photo detectors, photodiodes, line-scan and two-dimensional cameras, and photodiode arrays can be used to perform this detection function.

It should be noted that, as used herein, the term mechanism can encompass hardware, software, firmware, or any suitable combination thereof.

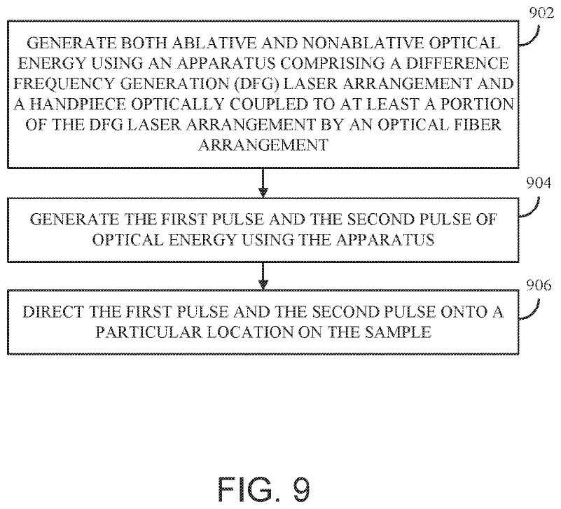

FIG. 9 shows an example 900 of a process for directing optical energy onto a sample in accordance with some embodiments of the disclosed subject matter. As shown in FIG. 9, at 902, process 900 can generate both ablative and nonablative optical energy using an apparatus comprising a difference frequency generation (DFG) laser arrangement and a handpiece optically coupled to at least a portion of the DFG laser arrangement by an optical fiber arrangement. In some embodiments, the DFG laser arrangement may be configured to generate both ablative and nonablative optical energy, and the handpiece may include at least one of an optical or a micromechanical element configured to deliver a first pulse and a second pulse of optical energy. At 904, process 900 can generate the first pulse and the second pulse of optical energy using the apparatus. Finally, at 906, process 900 can direct the first pulse and the second pulse onto a particular location on the sample. In various embodiments, a first amount of at least one of ablative optical energy or nonablative optical energy in the first pulse may be different from a second amount of at least one of ablative optical energy or nonablative optical energy in the second pulse.

It should be understood that the above-described steps of the process of FIG. 9 can be executed or performed in any order or sequence not limited to the order and sequence shown and described in the figures. Also, some of the above steps of the processes of FIG. 9 can be executed or performed substantially simultaneously where appropriate or in parallel to reduce latency and processing times.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof. It will thus be appreciated that those skilled in the art will be able to devise numerous systems, arrangements and methods which, although not explicitly shown or described herein, embody the principles of the invention and are thus within the spirit and scope of the present invention.

1. An apparatus for directing optical energy onto a sample, comprising:
a difference frequency generation (DFG) laser apparatus;
a handpiece optically coupled to at least a portion of the DFG laser apparatus by an optical fiber arrangement; and
a controller in operative communication with the DFG laser apparatus and the handpiece,
wherein the DFG laser apparatus is configured to generate both ablative and nonablative optical energy, and
wherein the handpiece comprises at least one optical or micromechanical element configured to generate a first pulse and a second pulse of optical energy,
wherein a first amount of at least one of ablative optical energy or nonablative optical energy in the first pulse is different from a second amount of at least one of ablative optical energy or nonablative optical energy in the second pulse, and
wherein the controller is configured to direct the first pulse and the second pulse onto a particular location on the sample using the handpiece.

2. The apparatus of claim 1, wherein the first pulse comprises substantially ablative optical energy and the second pulse comprises substantially nonablative optical energy.

3. The apparatus of claim 1, wherein the handpiece is configured to direct a plurality of pulses of energy onto a particular location on the sample, and wherein the plurality of pulses comprises at least one first pulse and at least one second pulse.

4. The apparatus of claim 1, wherein the DFG laser is configured to provide optical energy comprising a pump wavelength of 1030 nm, a seed wavelength of 1560 nm, and combined output wavelengths of 3050 nm and 3200 nm.

5. The apparatus of claim 1, wherein the DFG laser is configured to provide optical energy comprising a pump wavelength of 1030 nm, a seed wavelength of 1440 nm, and combined output wavelengths of 3600 nm and 2400 nm.

6. The apparatus of claim 1, wherein the DFG laser is configured to provide optical energy having a pump wavelength of 1064 nm, a seed wavelength of 1560 nm, and combined output wavelengths of 3350 nm and 2900 nm.

7. The apparatus of claim 1, wherein the at least one optical or micromechanical element comprises a polarizer and a polarizing beam splitter.

8. The apparatus of claim 1, wherein the at least one optical or micromechanical element comprises at least one of an electronic shutter and an optical chopper.

9. The apparatus of claim 1, wherein the at least one optical or micromechanical element comprises electronically-driven optical elements that include at least one of an acousto-optic modulator, an electro-optic modulator, flip/movable mirrors, electrical actuators, galvanometer scanners, rotation stages, or spinning mirror drums.

10. The apparatus of claim 1, wherein the apparatus is configured to perform at least one of a skin fractional surfacing procedure or a laser-assisted drug delivery procedure.

11. The apparatus of claim 1, wherein the controller, when directing the first pulse and the second pulse onto a particular location on the sample, is further configured to:
simultaneously direct both the first pulse and the second pulse onto the particular location on the sample.

12. The apparatus of claim 2, wherein the controller, when directing the first pulse and the second pulse onto a particular location on the sample, is further configured to:
direct the first pulse onto the particular location on the sample during a first time period, and
direct the second pulse onto the particular location on the sample during a second time period following the first time period.

13. The apparatus of claim 2, wherein the controller, when directing the first pulse and the second pulse onto a particular location on the sample, is further configured to:
direct the first pulse onto the particular location on the sample during a first time period, and
direct the second pulse onto the particular location on the sample during a second time period prior to the first time period.

14. The apparatus of claim 2, wherein the controller, when directing the first pulse and the second pulse onto a particular location on the sample, is further configured to:
alternate directing the first pulse and the second pulse onto the particular location on the sample during different time periods.

15. The apparatus of claim 1, wherein the controller, when directing the first pulse and the second pulse onto a particular location on the sample, is further configured to:
direct the first pulse onto the particular location on the sample during a first time period, and
following a delay, direct the second pulse onto the particular location on the sample during a second time period.

16. The apparatus of claim 1, wherein the controller, when directing the first pulse and the second pulse onto a particular location on the sample, is further configured to:
apply a stabilizer to the sample, and
direct the first pulse and the second pulse onto the stabilizer at the particular location on the sample.

17. The apparatus of claim 16, wherein the stabilizer comprises at least one of a plate, a film, or a mask.

18. The apparatus of claim 16, wherein the stabilizer comprises a mask including a plurality of openings therein, and
wherein the controller, when directing the first pulse and the second pulse onto a particular location on the sample, is further configured to:
direct the first pulse and the second pulse onto the particular location on the sample through at least one of the plurality of openings in the mask.

19. The apparatus of claim 16, wherein the stabilizer comprises a high thermal capacity material.

20. The apparatus of claim 16, wherein the stabilizer comprises a cooling system.

21. The apparatus of claim 1, wherein the particular location on the sample comprises a compound applied thereto.

22. The apparatus of claim 21, wherein the compound comprises a hydrophilic and low molecular weight compound.

23. The apparatus of claim 21, wherein the controller, when directing the first pulse and the second pulse onto a particular location on the sample, is further configured to:
image the sample using an imaging arrangement associated with the apparatus,
adjust a position of the apparatus relative to the sample using a position controller, and
direct the first pulse and the second pulse onto the particular location on the sample.

24. The apparatus of claim 1, wherein the sample comprises a biological tissue.

25-48. (canceled)
